(19) United States
(12) Patent Application Publication
(10) Pub. No.: US 2003/0008918 A1
(43) Pub. Date: Jan. 9, 2003

(54) Method of livestock sex discriminant birth and treating agent to be used therein

(76) Inventors: Tsuneharu Kurogi, Miyazaki-shi (JP); Yasumasa Kurogi, Miyazaki (JP)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(21) Appl. No.: 10/181,782

(22) PCT Filed: Jan. 22, 2001
(86) PCT No.: PCT/JP01/00384

(30) Foreign Application Priority Data
Jan. 21, 2000 (JP) 200013118

(51) Int. Cl. A61K 31/185

(52) U.S. Cl. 514/553

(57) ABSTRACT

A means of sex discriminant birth whereby the sex discriminant birth can be performed at such a high possibility as almost perfectly ensuring the desired sex discrimination. Sex discriminant birth can he achieved by treating ova or sperm with a solution of a mixture of a basic amino acid with an inorganic alkali salt in in vitro fertilization or in vivo fertilization.

### TECHNICAL FIELD

The present invention relates to a method for preselecting the sex of livestock. More particularly, the present invention relates to a method for preselecting the sex of livestock with extremely high probability in in vitro fertilization which obtains fertilized ova in vitro or artificial insemination which obtains fertilized ova in vivo, and to a treating agent used therefor.

### BACKGROUND ART

In the livestock industry, since it is very important to obtain a large number of genetically superior livestock, a means to obtain superior livestock has been known for a long time.

Moreover, since the sex of livestock has a significant meaning from an economical point of view in practice, establishment of technology which enables preselection of the sex of livestock has been demanded for a long time.

As a method for obtaining genetically superior livestock, in vitro fertilization, including providing a hormone treatment to superior female livestock to induce superovulation, retrieving unfertilized ova, fertilizing the unfertilized ova in vitro by spermatozoa of male livestock having superior ability, and transplanting the fertilized ova into another female livestock (recipient) has been known in recent years.

A method including artificially inseminating the unfertilized ova in vivo after the superovulation treatment to obtain a large number of fertilized ova, and transplanting the fertilized ova into another female livestock (recipient) has also been known.

The present inventors have found that the sex of the livestock can be preselected by using a basic amino acid and adjusting the concentration thereof, and applied for a patent (Japanese Patent Application Laid-open No. 6-181666). Specifically, the present inventors have established technology for artificially changing the male-to-female birth ratio of livestock, which is about 50:50 in a natural state.

However, since the above method cannot ensure complete preselection of the desired sex, an increase in the probability of preselection of the sex has been demanded.

### DISCLOSURE OF THE INVENTION

The present inventors have conducted further studies to solve the above problem. As a result, the present inventors have found that the probability of preselection of the sex can be increased by using an inorganic alkali salt together with a basic amino acid. This finding has led to the completion of the present invention.

Specifically, a first object of the present invention is to provide a method for preselecting the sex of livestock which comprises treating ova or spermatozoa with a mixed solution of a basic amino acid and an inorganic alkali salt in in vitro fertilization wherein ova are removed from the body, fertilized with spermatozoa, and transplanted into a uterus.

A second object of the present invention is to provide a method for preselecting the sex of livestock which comprises treating the uterus with a mixed solution of a basic amino acid and an inorganic alkali salt before artificial insemination.

Still another object of the present invention is to provide a treating agent used for the above methods.

### BRIEF DESCRIPTION OF THE DRAWINGS

FIG. 1 is a view showing an example of a method for carrying out the present invention.

1: Vagina

2: Uterine cervical canal

3: Uterine horn

4: Uterus

5: Periphery of external genitalia

6: Mixed solution injector

7: Sperm injector (Katheter)

8: Mark

9: Release hole

a: Base of uterine horn

b: Sperm release position

c: Mixed solution release position

FIG. 2 is a view showing an example of an injector advantageously used in the present invention.

11: Outer tube

12: Inner tube

13: Securing hook

14: Release hole

15: Mark

16: Straw

17: Stopcock

18: Injection rod

### BEST MODE FOR CARRYING OUT THE INVENTION

The method for preselecting the sex of livestock of the present invention may be applied to both in vitro fertilization technology and artificial insemination technology. In the method of the present invention, utilization of a basic amino acid and an inorganic alkali salt and their concentration in a solution are very important.

Specifically, in the case where it is desired to selectively obtain female livestock, it is necessary to set the concentration of the basic amino acid in the solution to about 0.3-20 percent by weight (hereinafter referred to as “wt %”) and set the concentration of the inorganic alkali salt to about 0.001-3.8 wt %. In the case where it is desired to selectively obtain male livestock, it is necessary to set the concentration of the basic amino acid in the solution to about 0.1-5 wt % and set the concentration of the inorganic alkali salt to about 0.001-4.3 wt %.

As the basic amino acid used to preselect the sex of livestock, any of four basic amino acids (arginine, lysine, ornithine, and citrulline) may be used.

As the inorganic alkali salt, sodium chloride, potassium chloride, calcium chloride, disodium hydrogenphosphate, potassium hydrogenphosphate, calcium sulfate, magnesium sulfate, potassium dihydrogenphosphate, sodium hydrogencarbonate, magnesium chloride, potassium hydroxide, and the like may be used.

The combination of the basic amino acid and the inorganic alkali salt differs depending upon whether to preselect either male or female. Specifically, in order to selectively obtain male livestock, for example, the concentration of arginine, which is the basic amino acid, is set to 5 wt % or less and an appropriate amount of inorganic alkali salt is combined therewith. In the case of using sodium chloride as the inorganic alkali salt, the concentration of sodium chloride is set to about 0.9-1.5 wt % (particularly preferably about 1.1 wt %) In the case of using potassium chloride, the concentration is set to about 0.04-2.9 wt % (particularly preferably about 1.8 wt %). In the case of using disodium hydrogenphosphate, the concentration is set to about 0.27-4.3 wt % (particularly preferably about 1.4 wt %).

In order to selectively obtain female livestock, for example, the concentration of arginine, which is the basic amino acid, is set to 0.3 wt % or more and an appropriate amount of inorganic alkali salt is combined therewith. In the case of using sodium chloride as the inorganic alkali salt, the concentration of sodium chloride is set to about 0.9 wt % or less (particularly preferably about 0.8 wt %). In the case of using calcium chloride, the concentration is set to about 0.02-2.2 wt % (particularly preferably about 0.1-0.3 wt %).

In the case of carrying out the present invention in the in vitro fertilization, either ova obtained by superovulation treatment or spermatozoa are treated with a mixed solution of the basic amino acid and the inorganic alkali salt (hereinafter called “mixed solution”) Fertilized ova obtained using the treated ova or spermatozoa are transplanted into another female livestock (recipient). In the case of using a mixed solution in which the concentration of either the basic amino acid or inorganic alkali salt is high, it is preferable to prepare the lowest concentration solution and gradually increase the concentration to a specific value.

In the case of treating spermatozoa with the mixed solution in the in vitro fertilization, spermatozoa are immersed in a solution prepared by diluting the basic amino acid and the inorganic alkali salt with a physiological saline solution or a semen diluent at a specific concentration. Ova are then fertilized by the spermatozoa. A period of time for immersing the spermatozoa in the mixed solution is about 1-4 minutes.

In the case of treating ova with the mixed solution, the basic amino acid and the inorganic alkali salt are added to an unfertilized ovum culture medium at a specific concentration, and the ova are cultured for a specific period of time and fertilized by spermatozoa. The treatment time is about 2-6 minutes.

In the case of carrying out the present invention in the artificial insemination, a position of the uterus to which spermatozoa are to be injected is treated with the mixed solution packed in a straw before artificial insemination, and ova are fertilized by injecting spermatozoa, for example.

The mixed solution used herein is a solution prepared by diluting the basic amino acid and the inorganic alkali salt with a semen diluent (“NF-3” manufactured by Takeda Chemical Industries, Ltd.), a physiological saline solution, or the like. In the case of treating a bovine uterus, the mixed solution is injected in an amount of about 0.5-1 ml, for example. The spermatozoa are injected 1-6 minutes after the treatment using the mixed solution. More specifically, the spermatozoa are injected 1-4 minutes after the treatment using the mixed solution for males. The spermatozoa are injected 2-6 minutes after the treatment using the mixed solution for females.

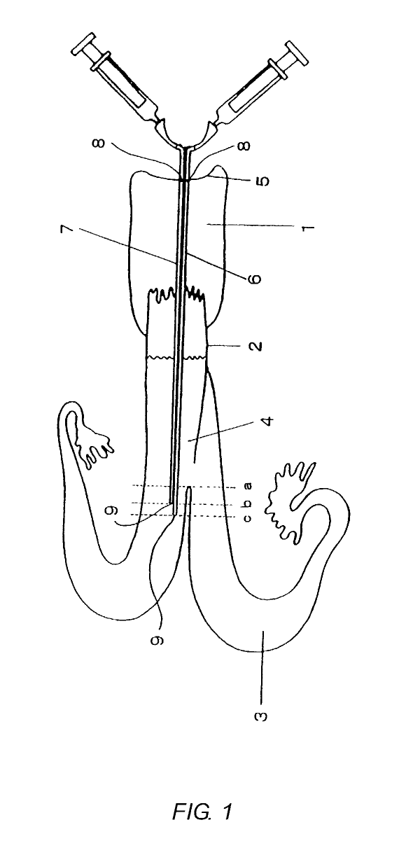

As a preferable example of the operation of the artificial insemination, as shown in FIG. 1, the mixed solution is injected into the uterus on the ovulation side (c), and the spermatozoa are injected into a position (b) located nearer the uterine cervical canal than the injected mixed solution.

Use of an injector to which a mark is attached so that the standard for determining the injection position is measured at the periphery of the external genitalia when injecting the mixed solution and spermatozoa using the above method enables the injection technology to be universal, whereby the probability of sex preselection can be improved.

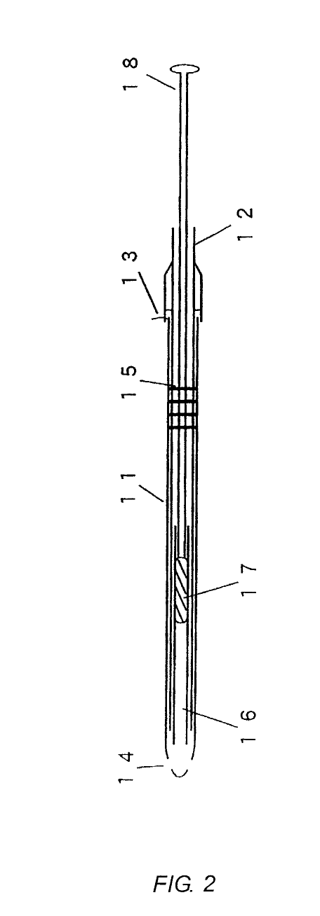

FIG. 2 shows an example of an injector advantageously used for the above object. In FIG. 2, 11 indicates an outer tube, 12 indicates an inner tube, 13 indicates a securing hook, 14 indicates a release hole, 15 indicates a mark, 16 indicates a straw, 17 indicates a stopcock, and 18 indicates an injection rod. This injector has a structure in which the outer tube 11 and the inner tube 12 are secured by the securing hook 13. When using the injector, the injection rod 18 is installed in the stopcock 17 of the straw 16 which is filled with the mixed solution (or semen), and placed in the inner tube. The injector is inserted at a specific position of the uterus of an animal in accordance with the mark 15 attached to the outer tube (or inner tube). The mixed solution (or semen) in the straw is injected to the specific position of the uterus from the release hole 14 by pressing the injection rod. Since this injector has the mark 15, the mixed solution (or semen) is always injected at the same position. This enables preselection of the sex of animals with high probability.

The above-described method of the present invention may be applied to mammals such as large animals such as cattle, pigs, horses, goats, sheep, and dogs, and small experimental animals such as cats, rabbits, mice, guinea pigs, and hamsters. According to the method of the present invention, animals having a desired sex can be selectively obtained.

The functional mechanism of the method for preselecting the sex of livestock of the present invention is not fully clarified. At the moment, it is considered that X spermatozoa are capacitated by a high concentration of basic amino acid and Y spermatozoa are capacitated by a low concentration of basic amino acid at the time of capacitation. This facilitates a conception by either the X spermatozoa or Y spermatozoa and produces an inclination toward the specific sex. The effect of the basic amino acid is considered to be increased by the inorganic alkali salt.

### EXAMPLES

The present invention is described below in more detail by examples, which should not be construed as limiting the present invention.

### Example 1

(1) Control of Sex by Treatment of Spermatozoa Using Basic Amino Acid and Inorganic Alkali Salt:

Arginine and calcium chloride were added to a semen diluent “NF-3” (manufactured by Takeda Chemical Industries, Ltd.) so that the concentration was respectively 13 wt % and 0.22 wt % to prepare a mixed solution for female. Bovine spermatozoa were immersed in 0.5 ml of this solution for 1-4 minutes. Bovine ova were fertilized by the bovine spermatozoa to obtain fertilized ova. These fertilized ova were transplanted into a female recipient. Bovine spermatozoa were immersed in a semen diluent containing 3 wt % of arginine, 1.1 wt % of sodium chloride, 1.8 wt % of potassium chloride, and 1.4 wt % of disodium hydrogenphosphate (mixed solution for male). Ova fertilized by the spermatozoa were transplanted into a recipient. The sex of a newborn calf delivered from the recipient was judged. The results are shown in Table 1.

(2) Control of Sex by Treatment of Ova Using Basic Amino Acid and Inorganic Alkali Salt:

Unfertilized bovine ova were cultured in an ovum culture solution (“TCM-19” manufactured by Gibco) containing 13 wt % of arginine and 0.22 wt % of calcium chloride. The ova were fertilized by bovine spermatozoa to obtain fertilized ova. The fertilized ova were transplanted into a female recipient. The sex of a newborn calf delivered from the recipient was judged. Unfertilized bovine ova were cultured in an ovum culture solution containing 3 wt % of arginine, 1.1 wt % of sodium chloride, 1.8 wt % of potassium chloride, and 1.4 wt % of disodium hydrogenphosphate. The ova were fertilized by bovine spermatozoa to obtain fertilized ova. The fertilized ova were transplanted into a female recipient. The results are shown in Table 1.

(Results)

TABLE 1

| | | Female | Male |
|---|---|---|---|
| | Number of | birth | birth |
| Item | samples | rate (%) | rate (%) |
| Control group | 108 | 49 | 51 |
| Spermatozoon treatment | | | |
| Group of mixed solution for females | 60 | 92 | 8 |
| Group of mixed solution for males | 71 | 5 | 95 |
| Ovum treatment | | | |
| Group of medium for females | 51 | 89 | 11 |
| Group of medium for males | 55 | 8 | 92 |

As is clear from the above results, 92% of the newborn calves were female in the group in which the spermatozoa were immersed in the mixed solution for females. In the group in which the spermatozoa were immersed in the mixed solution for males, 95% of the newborn calves were male. In the case where the ova were cultured in the culture medium for females, 89% of the newborn calves were female. In the case where the ova were cultured in the culture medium for males, 92% of the newborn calves were male.

This shows that the sex of the fertilized ova can be selected by allowing the spermatozoa or ova to be in contact with the mixed solution of the basic amino acid and the inorganic alkali salt at various concentrations.

Similar results were obtained in the case of using lysine, ornithine, or citrulline instead of arginine.

### Example 2

Sex Preselection in in vivo Fertilization:

Arginine and calcium chloride were added to a semen diluent “NF-3” so that the concentration was respectively 13 wt % and 0.22 wt % to prepare a mixed solution for females. Arginine, sodium chloride, potassium chloride, and disodium hydrogenphosphate were added to a semen diluent “NF-3” so that the concentration was respectively 3 wt %, 1.1 wt %, 1.8 wt %, and 1.4 wt % to prepare a mixed solution for males. Each of the mixed solutions was packed in a 0.5-1 ml straw for artificial insemination and stored in a freezer at −20° C. or at −196° C. (liquid nitrogen) to prepare a treating solution for females and a treating solution for males.

The treating solution for males was injected into the uterus 1-4 minutes before artificial insemination of a bovine. The treating solution for females was injected into the uterus 2-6 minutes before artificial insemination. Artificial insemination was carried out according to a conventional method, and the sex of a newborn calf was judged. The results are shown in Table 2.

(Results)

TABLE 2

| | | Female | Male |
|---|---|---|---|
| | Number of | birth | birth |
| Item | samples | rate (%) | rate (%) |
| Control group | 108 | 49 | 51 |
| Group of treating solution for | 155 | 92 | 8 |
| females | | | |
| Group of treating solution for | 252 | 4 | 96 |
| males | | | |

As is clear from these results, the female-to-male birth ratio in the control group was approximately 1:1 (female: 49%, male: 51%). However, in the case of treating the bovine uterus with the mixed solution for females, 92% of the newborn calves were female. In the case of treating the bovine uterus with the mixed solution for males, 96% of the newborn calves were male.

### Example 3

Sex Preselection Using Frozen Semen for Artificial Insemination:

Bovine semen was treated with a semen diluent to which arginine and calcium chloride were added in advance so that the concentration was respectively 13 wt % and 0.22 wt % according to a conventional method to prepare frozen bovine semen for artificial insemination (for females). Frozen bovine semen for artificial insemination (for males) was prepared using a semen diluent containing 3 wt % of arginine, 1.1 wt % of sodium chloride, 1.8 wt % of potassium chloride, and 1.4 wt % of disodium hydrogenphosphate. Artificial insemination was carried out using the above frozen semen, and the male-to-female birth ratio was examined. The results are shown in Table 3.

(Results)

TABLE 3

| | | Female | Male |
|---|---|---|---|
| | Number of | birth | birth |
| Item | samples | rate (%) | rate (%) |
| Control group | 168 | 48 | 52 |
| Group of frozen semen for females | 120 | 83 | 17 |
| Group of frozen semen for males | 153 | 15 | 85 |

As is clear from these results, the female-to-male birth ratio in the control group was 48:52. However, in the group in which the frozen semen for females was used, the birth rate of females was as high as 83%. In the group in which the frozen semen for male was used, the birth rate of males was as high as 85%.

### INDUSTRIAL APPLICABILITY

According to the present invention, the sex preselection can be achieved with high probability in in vivo and in vitro fertilization by the treatment using a mixed solution of the basic amino acid and inorganic alkali salt.

Therefore, animals having genetically superior characters can be efficiently produced at low cost. Because of this, this technology is very advantageous in the livestock industry and the field of life science.

1. A method for preselecting the sex of livestock which comprises treating ova or spermatozoa with a mixed solution of a basic amino acid and an inorganic alkali salt in in vitro fertilization wherein ova are removed from the body, fertilized with spermatozoa, and transplanted into a uterus.

2. The method according to claim 1, wherein ova or spermatozoa are treated with a mixed solution containing 0.3-20 wt % of a basic amino acid and 0.001-3.8 wt % of an inorganic alkali salt, thereby selectively obtaining female livestock.

3. The method according to claim 1, wherein ova or spermatozoa are treated with a mixed solution containing 0.1-5 wt % of a basic amino acid and 0.001-4.3 wt % of an inorganic alkali salt, thereby selectively obtaining male livestock.

4. A method for preselecting the sex of livestock which comprises treating the uterus with a mixed solution of a basic amino acid and an inorganic alkali salt before artificial insemination.

5. The method according to claim 4, wherein the uterus is treated with a mixed solution containing 0.3-20 wt % of a basic amino acid and 0.001-3.8 wt % of an inorganic alkali salt, thereby selectively obtaining female livestock.

6. The method according to claim 4, wherein the uterus is treated with a mixed solution containing 0.1-5 wt % of a basic amino acid and 0.001-4.3 wt % of an inorganic alkali salt, thereby selectively obtaining male livestock.

7. The method according to any one of claims 1 to 6, wherein the basic amino acid is arginine, lysine, ornithine, or citrulline.

8. The method according to any one of claims 1 to 6, wherein the inorganic alkali salt is sodium chloride, potassium chloride, calcium chloride, disodium hydrogenphosphate, potassium hydrogenphosphate, calcium sulfate, magnesium sulfate, potassium dihydrogenphosphate, sodium hydrogencarbonate, magnesium chloride, or potassium hydroxide.

9. A treating agent for female livestock selection birth, which is prepared by adding a basic amino acid and an inorganic alkali salt to a semen diluent or a physiological saline solution so that the concentration is respectively 0.3-20 wt % and 0.001-3.8 wt %.

10. A treating agent for male livestock selection birth, which is prepared by adding a basic amino acid and an inorganic alkali salt to a semen diluent or a physiological saline solution so that the concentration is respectively 0.1-5 wt % and 0.001-4.3 wt %.

11. An ovum treating agent for female livestock selection birth, which is prepared by adding a basic amino acid and an inorganic alkali salt to an ovum culture medium so that the concentration is respectively 0.3-20 wt % and 0.001-3.8 wt %.

12. An ovum treating agent for male livestock selection birth, which is prepared by adding a basic amino acid and an inorganic alkali salt to an ovum culture medium so that the concentration is respectively 0.1-5 wt % and 0.001-4.3 wt %.

13. The treating agent according to any one of claims 9 to 12, wherein the basic amino acid is arginine, lysine, ornithine, or citrulline.

14. The treating agent according to any one of claims 9 to 12, wherein the inorganic alkali salt is sodium chloride, potassium chloride, calcium chloride, disodium hydrogenphosphate, potassium hydrogenphosphate, calcium sulfate, magnesium sulfate, potassium dihydrogenphosphate, sodium hydrogencarbonate, magnesium chloride, or potassium hydroxide.

15. A frozen semen for artificial insemination for female, which is prepared by using a semen diluent to which a basic amino acid and an inorganic alkali salt are added so that the concentration is respectively 0.3-20 wt % and 0.001-3.8 wt %.

16. A frozen semen for artificial insemination for male, which is prepared by using a semen diluent to which a basic amino acid and an inorganic alkali salt are added so that the concentration is respectively 0.1-5 wt % and 0.001-4.3 wt %.

17. A treating agent injector including an inner tube and an outer tube which is used to inject a treating agent into the uterus of an animal from the inside of the inner tube, wherein the inner tube has open ends, the outer tube has a release hole at a front end and an open rear end, a mark indicating an insertion position into the uterus is attached to the tube section, and the inner tube and the outer tube are combined near the rear ends thereof.

18. The treating agent injector according to claim 17, wherein a plurality of marks is attached to the outer tube and/or inner tube.
